(19) United States
(12) Patent Application Publication
(10) Pub. No.: US 2020/0041496 A1
(43) Pub. Date: Feb. 6, 2020

(54) Methods for Identifying and Treating Mammalian Subjects with Insulin Resistance

(71) Applicant: Joshua Health Care LLC, Omaha, NE (US)

(72) Inventors: David E. Kershner, Omaha, NE (US); Vasileios Margaritis, Attiki (GR)

(21) Appl. No.: 16/528,215

(22) Filed: Jul. 31, 2019

Related U.S. Application Data
(60) Provisional application No. 62/713,064, filed on Aug. 1, 2018.
(60) Provisional application No. 62/801,500, filed on Feb. 5, 2019.

(51) Int. Cl. G01N 33/53; G01N 33/50

(52) U.S. Cl. CPC G01N 33/5308; G01N 33/5091; G01N 2800/50; G01N 2800/56; G01N 2800/042

(57) ABSTRACT

A method of diagnosing and treating insulin resistance at the end of a first phase insulin response to glucose in a mammalian subject is disclosed. The method includes administering an amount of glucose to the mammalian subject capable of producing a first phase insulin response to glucose. At the end of the first phase insulin response to glucose, a serum sample is obtained from the mammalian subject, from which the levels of at least one biomarker for insulin resistance may be measured, from a group including insulin, proinsulin, C-peptide, glucose, and hemoglobin A1c. An analysis of the levels of the biomarkers in the serum samples may be performed to determine if the mammalian subject is insulin dependent. If the mammalian subject is determined to have insulin resistance, treatments for insulin resistance may be prescribed. Prescribed treatments include weight loss, dietary modification, exercise, or drug therapy.

### CROSS-REFERENCE TO RELATED APPLICATION

The present application claims the benefit under 35 U.S.C. § 119(e) of U.S. Provisional Application Ser. No. 62/713,064 filed Aug. 1, 2018, entitled GLUCOSE INSULIN SECRETION TEST FOR IDENTIFYING PATIENTS WITH INSULIN RESISTANCE, and U.S. Provisional Application Ser. No. 62/801,500, filed Feb. 10, 2019, entitled ORAL GLUCOSE INSULIN SECRETION TEST FOR IDENTIFYING PATIENTS WITH INSULIN RESISTANCE, naming David, E. Kershner and Vasileios Margaritis as inventors, which is hereby incorporated by reference in its entirety.

### TECHNICAL FIELD

This invention relates to methods for diagnosing insulin resistance in a mammalian subject, and more particularly to using blood-based biomarkers and their combinations from blood samples collected during the first phase insulin response to diagnose insulin resistance in a mammalian subject.

### BACKGROUND

Insulin resistance (IR) is characterized by the reduced response of target tissue to the polypeptide hormone insulin. The reduced response of the tissue to insulin is the major pathogenesis of Type 2 diabetes mellitus. Insulin is produced by the pancreatic β-cells and is responsible for glucose uptake by tissues. Although the mechanism of IR is complex, the reduced skeletal muscle sensitivity to insulin is the primary defect leading to impaired glucose synthesis and insulin resistance. the reduced sensitivity of insulin by target cells results in a normal increased secretion of insulin by the β-cells to maintain glucose homeostasis. As the muscle tissue further becomes insulin resistant, postprandial glucose plasma concentrations rise further. This, in turn, further stimulates insulin response to maintain glucose levels. The stimulated oversecretion of insulin promotes pancreatic β-cell exhaustion/death with the progression of time, which results in chronic hyperglycemia and diabetes.

Increased insulin release in IR patients results in increased free fatty acid release by adipocytes, which in turn stimulates the upregulation of many proinflammatory markers, including PAI-1, C-reactive protein, ferritin, fibrinogen, homocysteine, interleukin-6, Lipoprotein(a), and tumor necrosis factor alpha. This proinflammatory state in IR patients also potentiates cardiovascular disease, particularly atherosclerosis. Biomarkers related to inflammatory associated atherosclerosis include C-reactive protein, BNP, Lipoprotein(a), total cholesterol, LDL, HDL, Triglycerides and non-HDL cholesterol. Persistent IR is also associated with liver disease. The damage to liver tissue in IR patients can be assessed by measuring levels of liver enzymes, including alanine transaminase, aspartate aminotransferase, and alkaline phosphatase. When assessed along with the diagnosis of IR, these biomarkers help to confirm the veracity of the IR diagnosis and illuminate pathogenic processes that have initiated in the IR patient.

Currently, there is no relatively inexpensive, standardized test for the identification of IR in the context of clinical practice. The euglycemic insulin clamp test is considered to be the best available benchmark test for the measuring insulin secretion and insulin action in vivo. This technique requires the patient to be hospitalized overnight using the administration of intravenous glucose to maintain serum glucose levels at the euglycemic concentration. The advantage is that is test primarily reflects the uptake of skeletal muscle, which is significant for evaluating IR during the first phase insulin response. The disadvantage of this test is the invasiveness requiring a hospital stay and significant cost involved. These disadvantages prohibit the test to be used in the clinic.

Other tests for IR include the insulin suppression test, oral glucose tolerance test, insulin tolerance test, insulin suppression test, and the frequently sampled intravenous glucose tolerance test with minimal model analysis. These tests are also invasive with the tests ranging from 2-5 hours in length and have a considerable cost involved. Although these tests provide data on skeletal muscle sensitivity to insulin, they also provide secondary hepatic insulin sensitivity and glucose production. Because of the importance of identifying IR and the disadvantages of this tests, surrogate tests were developed which include the homeostasis model (HOMA) and quantitative insulin-sensitivity check index (QUICKI). These tests can be performed under clinic conditions for patient diagnosis but are based on mathematical fasting insulin and glucose and are unable to generate data for skeletal muscle insulin sensitivity.

By the time impaired glucose tolerance or impaired fasting glucose (IFG) is elevated a significant amount of β-cell exhaustion has occurred. It would then seem likely that identifying IR early in the individual to preserve β-cell function would be more successful in preventing diabetes than after cell injury has occurred. This problem creates the need for a method to identify IR in the individual for both preventative diagnosis and evaluation of treatment in those with the condition.

### SUMMARY

A method of diagnosing and treating insulin resistance at the end of a first phase insulin response to glucose in a mammalian subject is disclosed, in accordance with one or more embodiments of the present disclosure. In some embodiments, the method comprises: administering an amount of glucose to the mammalian subject capable of producing a first phase insulin response to glucose; obtaining a blood sample from the mammalian subject at the end of the first phase insulin response to glucose; isolating serum from the blood sample; determining the serum concentration of at least one biomarker for insulin resistance from a group of biomarkers comprising insulin, proinsulin, C-peptide, glucose, and hemoglobin A1c in the serum sample; performing an analysis of the levels of the at least one biomarker for insulin resistance in the serum sample to determine if the mammalian subject is insulin resistant, wherein the analysis includes comparing the determined levels of the at least one biomarker in the sample to insulin resistance-positive and insulin resistance-negative reference levels; and administering a treatment to the mammalian subject if the mammalian subject is determined to be insulin resistant, wherein the treatment comprises at least one of weight loss, dietary modification, exercise, or drug therapy, wherein the drug therapy is an effective amount of at least one of an insulin, a metformin, a thiazolidinedione, or a sulfonylurea.

A method of diagnosing insulin resistance at the end of a first phase insulin response to glucose in a mammalian subject is disclosed, in accordance with one or more embodiments of the present disclosure. In one embodiment, the method comprises: administering an amount of glucose to the mammalian subject capable of producing a first phase insulin response to glucose; obtaining a blood sample from the mammalian subject at the end of the first phase insulin response to glucose; isolating serum from the blood sample; determining the serum concentration of at least one biomarker for insulin resistance from a group of biomarkers comprising insulin, proinsulin, C-peptide, glucose, or hemoglobin A1c in the serum sample; and performing an analysis of the levels of the at least one biomarker for insulin resistance in the serum sample to determine if the mammalian subject is insulin resistant, wherein the analysis includes comparing the determined levels of the at least one biomarker in the sample to insulin resistance-positive and insulin resistance-negative reference levels.

It is to be understood that both the foregoing general description and the following detailed description are exemplary and explanatory only and are not necessarily restrictive of the present disclosure. The accompanying drawings, which are incorporated in and constitute a part of the specification, illustrate subject matter of the disclosure. Together, the descriptions and the drawings serve to explain the principles of the disclosure.

### BRIEF DESCRIPTION OF THE DRAWINGS

The numerous advantages of the disclosure may be better understood by those skilled in the art by reference to the accompanying figures in which:

FIG. 1 is a flow diagram illustrating a method for diagnosing and treating a mammalian subject with insulin resistance, in accordance with one or more embodiments of this disclosure.

FIG. 2 is a high-level flowchart illustrating a method for determining insulin binding to target tissues and insulin production by pancreatic β-cells, in accordance with one or more embodiments of this disclosure.

FIG. 3A is a scatterplot analysis illustrating the relationship between HbA1c and age, in accordance with one or more embodiments of this disclosure.

FIG. 3B is a scatterplot analysis illustrating the relationship between insulin production and age, in accordance with one or more embodiments of this disclosure.

### DETAILED DESCRIPTION

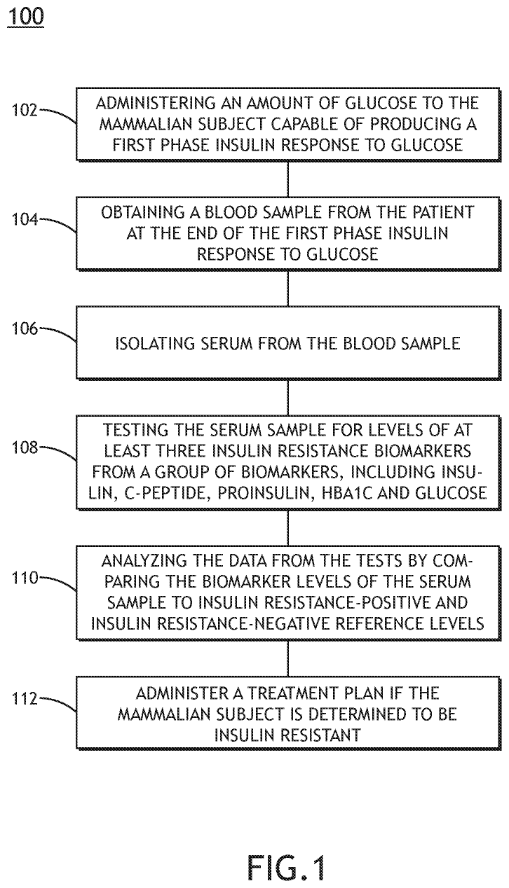

Reference will now be made in detail to the subject matter disclosed, which is illustrated in the accompanying drawings. Referring generally to FIGS. 1 through 3B, a method diagnosing and treating insulin resistance at the end of a first phase insulin response to glucose in a mammalian subject is described in accordance with the present disclosure.

Embodiments of the present disclosure are directed to diagnosing insulin resistance at the end of a first phase insulin response to glucose. The first phase insulin response is a rapid release of insulin that is triggered in response to increased blood glucose levels. It is noted that a variety of biomarkers may be used for diagnosing insulin resistance, including, but not limited to, insulin, proinsulin, C-peptide, and hemoglobin A1c. These biomarkers may be analyzed along with other biomarkers of disease related to insulin resistance and diabetes. Additional embodiments of the present disclosure are directed to treatment of insulin resistance based upon the diagnosis of insulin resistance as determined by the analysis of biomarkers obtained at the end of a first phase insulin response to glucose.

FIG. 1 illustrates a method 100 for the diagnosis and treatment of insulin resistance in a mammalian subject. In step 102, the method 100 includes administering a specified amount of glucose to the mammalian subject capable of producing a first phase insulin response to glucose. The administration of glucose may be achieved by any method known in the art for administering glucose, including, but not limited to, intravenous, sublingual, intranasal, enteral, and oral administration.

The administration of glucose to the mammalian subject further includes any consumable form of glucose known in the art for use in insulin resistance testing, including, but not limited to, chewable tablets, glucose gel, injectable glucose, and a drinkable liquid.

The amount of glucose administered to the mammalian subject is any amount that would invoke a first phase insulin response to glucose. The amount of glucose administered is dependent on the species and weight of the mammalian animal tested and can range from 1 to 1000 grams of glucose. For example, the range of glucose administered may be 29 to 37 grams. By way of another example, the amount of glucose administered may be 36 grams.

In some embodiments, the mammalian subject fasts (e.g., does not eat food or drink caloric beverages) before the glucose is administered. The amount of time fasting is dependent on the species of the mammalian animal tested and can range from 0 hours to 24 hours before the administration of glucose. In an embodiment, the amount of time for fasting before glucose administration is approximately eight hours.

In step 104, method 100 includes obtaining a blood sample from the patient at the end of the first phase insulin response to glucose. Blood samples may be obtained from the patient by any means known in the art, including, but not limited to, finger stick, intravenous line sampling, and venipuncture.

The time course of the first phase insulin response to glucose may differ between mammalian species, populations, and individuals. Therefore, the time that a blood sample is obtained after first phase insulin response has ended may vary. In some embodiments, the blood sample may be obtained from 5 minutes to 20 minutes after administration of glucose. In an embodiment, the blood sample may be obtained 15 minutes after administration of glucose.

In step 106 of the method 100, serum is isolated from the blood sample. Serum is the liquid, noncellular component of blood, typically isolated after the cells in the blood sample have clotted. The serum may be isolated from the blood sample by any method known in the art, including, but not limited to, centrifugation, filtration, and through chromatographic separation.

In step 108 of the method 100, the levels of insulin resistance-related biomarkers in the serum sample may be tested. The number of insulin resistance biomarkers to be tested may range from one to as many as 20. In some embodiments, one insulin resistance biomarker may be tested. In some embodiments, three insulin resistance biomarkers may be tested. The insulin resistance-related biomarkers to be tested include, but are not limited to, insulin, proinsulin, C-peptide, glucose, and Hemoglobin A1c (HbA1c).

The method 100 for diagnosis and treating insulin resistance may further include testing the serum sample for levels of inflammation markers in the serum sample. Chronic inflammation is often associated with insulin resistance. Biomarkers for chronic inflammation include, but are not limited to PAI-1, C-reactive protein, ferritin, fibrinogen, homocysteine, interleukin-6, and Lipoprotein(a).

The method 100 for diagnosis and treating insulin resistance may further include testing the serum sample for biomarkers of cardiovascular disease. Cardiovascular disease is often associated with insulin resistance. Biomarkers for cardiovascular disease include, but are not limited to, C-reactive protein, BNP, Lipoprotein (a), total cholesterol, LDL, HDL, Triglycerides and non-HDL cholesterol.

The method 100 for diagnosis and treating insulin resistance may further include testing the serum sample for biomarkers of liver damage. Liver damage is often associated with insulin resistance. Biomarkers for cardiovascular disease include, but are not limited to, alanine transaminase, aspartate aminotransferase, alkaline phosphatase and free fatty acids.

The method 100 for diagnosis and treating insulin resistance may further include measurements of the mammalian subject that are not derived from the serum sample. For instance, physical measurements of the mammalian subject may include, but are not limited to, weight, body mass index (BMI), height, and skin fold test. In some embodiments, measurements not derived from serum sample may include biochemical analysis of urine.

In step 110 of the method 100, the data from the tested biomarkers are analyzed by comparing the biomarker levels of the serum sample to insulin resistance-positive and insulin resistance-negative reference levels. The analysis 110 may further measurements or characteristics (e.g., risk factors) of the mammalian subject other than those found in the blood sample, including, but not limited to, age, weight, BMI, sex, and ethnicity. The analysis 110 may be performed by any method known in the art for performing analysis, including manual comparison of reference levels to the sample levels and using a computer to compare reference levels to the sample levels (e.g., using statistical software or spreadsheet software).

In step 112 of the method 100, a treatment plan is administered if the mammalian subject is determined to be insulin resistant. Alternatively, the method may omit steps for the treatment of insulin resistance. Treatment for insulin resistance may include any type of treatment known in the art for treatment of insulin resistance, including, but not limited to, dietary modification, exercise, drug therapy and weight loss.

Prescribed dietary modification (e.g., a diet), may include any type of prescribed dietary modification known in the art for treating insulin resistance. For instance, the prescribed dietary modification may include an adjustment in caloric intake (e.g., lower caloric intake). The prescribed dietary modification may also include a reduction in carbohydrates or fats. The prescribed dietary modification may further include an increase in vegetables and fiber, as well as other foods known to reduce insulin resistance.

Prescribed exercise may include any type of prescribe exercise routine or plan designed for the reduction and treatment of insulin resistance in a mammalian subject. The prescribed exercise routine or plan may include any exercise routine or plan known in the art to reduce insulin resistance, including, but not limited to, low intensity work-outs, high-intensity work-outs, weight training, stretching, walking, cycling, swimming, and running.

Prescribed drug therapy for treatment of insulin resistance in a mammalian subject further includes any prescribed drug known in the art to reduce insulin resistance in a mammalian subject or reduces symptoms of insulin resistance in a mammalian subject. Drugs used to treat insulin resistance or reduce symptoms of insulin resistance include, but are not limited to, metformin, insulin (e.g., native and recombinant versions), thiazolidniediones (e.g., pioglitazone or rosiglitazone), and sulphonylureas (e.g., glimepiride, and/or gilbenclamide).

Prescribed weight loss therapy for treatment of insulin resistance in a mammalian further includes any prescribed weight loss therapies or regimens known in the art to reduce weight. Prescribed weight loss therapies or regiments include, but are not limited to, prescribed dietary modifications (e.g., caloric reduction), prescribed exercise (e.g., low intensity workouts, and/or walking), and prescribed weight loss medications (e.g., orlistat, lorcaserin, phentermine-topiramate, naltrexone-bupropion and/or liraglutide).

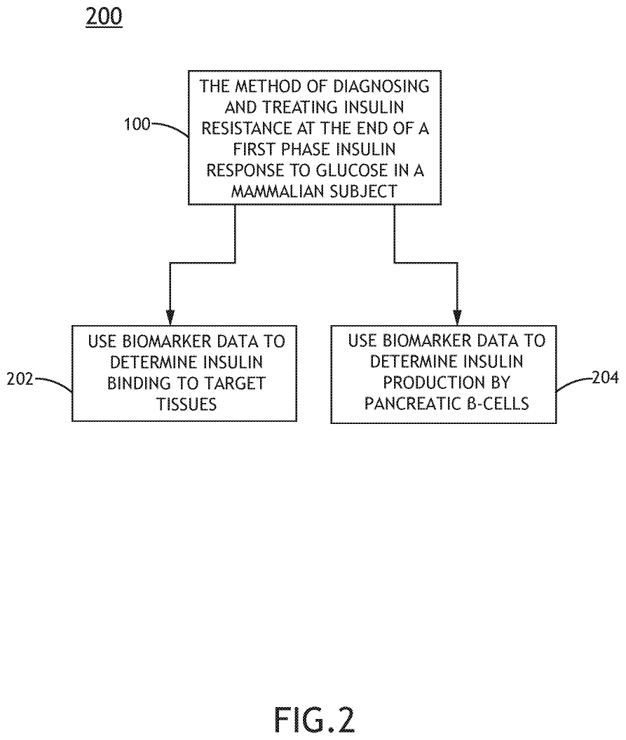

FIG. 2 is a high-level flowchart illustrating a method for determining insulin binding to target tissues and insulin production by pancreatic β-cells 200, in accordance with one or more embodiments of this disclosure. Biomarkers assayed in the method 100 for diagnosing and treating insulin resistance may be helpful in determining particular aspects of insulin signaling in the mammalian subject that are not functioning correctly.

In step 202 of method 200, biomarker data is used to determine insulin binding to target tissues. Biomarkers analyzed to determine the ability of insulin to bind the insulin receptor in tissues include, but are not limited to, insulin and proinsulin. A specific diagnosis and possible treatment may be prescribed on the basis of the inability of insulin to bind the insulin receptor. Prescribed treatments may include, but are not limited to, weight loss, dietary modification, exercise, and drug therapy therapies as described herein.

In step 204 of method 200, biomarker data is used to determine insulin production by pancreatic β-cells. Higher than normal production of insulin by the pancreatic β-cell may indicate insulin resistance, whereas lower than normal production of insulin by the pancreatic β-cell may indicate pancreatic β-cell exhaustion, a symptom of diabetes. Biomarkers analyzed to determine insulin production by pancreatic β-cells include, but are not limited to insulin, proinsulin, or C-peptide. In some embodiments, a treatment regimen may be prescribed based at least partially upon the ability of the pancreatic β-cells in a mammalian subject to produce and secrete insulin. Prescribed treatments may include, but are not limited to, weight loss, dietary modification, exercise, and drug therapy therapies as described herein.

The following examples are intended only to further illustrate the invention and are not intended to limit the scope of the subject matter which is defined by the claims.

### EXAMPLES

### Materials and Methods

IRB approval from Walden University was obtained prior to collecting any data for this study (IRB Approval Number: 12-05-16-0077958). A study coordinator nurse was trained on the study protocol and was responsible for identifying patients that met the criteria for metabolic syndrome who were then tested using the oral glucose insulin sensitivity test (OGIST). Patient data was obtained from patient charts, an electronic medical records system (EMR), and the Laboratory Information System (LIS) 18 months prior to the study. OGIST results were obtained 3 weeks after approval of the study by the IRB. The International Classification of Disease Version 10 codes used in the identification of patients meeting the criteria for this study included E16.3 (increased secretion of glucagon), E16.8 (other specific disorders of pancreatic secretion), E74.8 (other specific disorders of carbohydrate metabolism), and E88.81 (metabolic syndrome).

The EMR numeric patient identifier was maintained to allow access of the files by the granting medical center, and all patient information, such as patient's name, was disallowed. Variables were imported into an Excel 2010 spreadsheet and then imported into the SPSS Statistics 22 program for analysis. Variables included in the SPSS program for analysis comprises age, gender, ethnicity, BMI, insulin, proinsulin, C-peptide, HbA1c, and triglycerides. The descriptive analysis of the patients' data found that the study population comprised of White 50.4%, Hispanic 43.7%, Black 2.0%, and Asian 2.8% (see Table 1).

TABLE 1
Descriptive Statistics for Ethnicity and Percentages

| | | | | Valid |
|---|---|---|---|---|
| | | Frequency | Percent | Percent |
| Valid | Asian | 7 | 2.8 | 2.8 |
| | Black | 5 | 2.0 | 2.0 |
| | White | 128 | 50.4 | 51.0 |
| | Hispanic | 111 | 43.7 | 44.2 |
| | Total | 251 | 98.8 | 100.0 |
| Missing | System | 3 | 1.2 | |
| Total | | 254 | 100.0 | |

The gender demographics for this study equaled 131 women (51.6%) and 120 men (47.2%; see Table 2). The patient lab results used in this study included glucose, HbA1c, insulin, proinsulin, C-peptide, and triglyceride cholesterol levels. These results were obtained at the end of the first phase insulin response to glucose after a 36-gram glucose oral bolus was given to the patient. Patient results are included in Table 6 below.

The OGIST is given to those patients that initially meet the criteria for metabolic syndrome or have been previously diagnosed with IR and are under treatment. Patients were initially given 36 grams of oral concentrated glucose, (EASYDEX-100 BVO free: Aero-Med LTD.), and at 15 minutes (the end of the first phase insulin response to glucose) insulin, glucose, C-peptide, proinsulin, HgA1c levels are obtained. Those patients responding normally to glucose will demonstrate normal levels of insulin, glucose, proinsulin, and HgA1c. Descriptions of these biomarkers are detailed in table 3. Descriptions of independent variables used in this study are detailed in table 4. C-peptide will remain elevated since this peptide is secreted in 30-40 minutes by the kidneys. In those patients with IR, cellular sensitivity to insulin is reduced due to poor binding of insulin to the cellular insulin receptor. This results in higher levels of insulin, glucose, and proinsulin at the end of the first phase insulin response to glucose. Patients with advanced IR, insulin, proinsulin, and C-peptide will start to drop due to β-cell exhaustion, while glucose and HbA1c levels will increase based on both the sensitivity of insulin to the cell and the ability of the β-cell to produce insulin to maintain glucose homeostasis. Glucose and HbA1c levels were analyzed using a Beckman Coulter UniCel DxC800 chemistry analyzer. Insulin, proinsulin, and C-peptide were analyzed by chemiluminescent immunoassay on a Centaur/Centaur XP immunoassay platform. SPSS version 18.0 analytics software was used to analyze the data in this study.

TABLE 2
Descriptive Statistics for Gender and Percentages

| | | | | Valid |
|---|---|---|---|---|
| | | Frequency | Percent | Percent |
| Valid | Male | 120 | 47.2 | 47.8 |
| | Female | 131 | 51.6 | 52.2 |
| | Total | 251 | 98.8 | 100.0 |
| Missing | | 3 | 1.2 | |
| Total | | 254 | 100.0 | |

The surrogate indices of HOMA, QUICKI, and McA from the patient results were used for validation comparison with the results from the OGIST. The variables used in the comparisons, and the surrogate test used for each variable, are listed on table 5. The equations for surrogate indexes calculations for insulin sensitivity and IR included the following HOMA equation:

$$H O M A = \frac{fasting insulin \times fasting glucose}{22.5}$$

where the denominator 22.5 is the normalization factor initially calculated by testing normal healthy individuals. This model calculates the steady-state insulin and glucose concentrations. The following QUICKI equation:

$$QUICKI = \frac{1}{[ log ( Insulin µU \text{/} mL ) + ( log ( Glucose mg \text{/} dL ) ]}$$

mathematically converts fasting insulin and glucose levels using the logarithm and reciprocal of both. The McA equation is McA=exp[2.63-0.28 log(Insulin μU/mL)−0.31 log(Triglycerides mmol/L)] and uses both insulin and triglycerides levels to predict insulin sensitivity since insulin is the feedback hormone for triglyceride production.

A total of 251 patients with IR, either newly diagnosed or in treatment were included in this study, with a mean age of 49.2 and a mean BMI of 34. Because these patients had confirmed IR, it was reasonable to expect a higher BMI than normal since insulin levels higher than 25 mU/L stimulate adipogenesis during periods of hyperinsulinemia at the end of the first phase insulin response to glucose. The descriptive statistics for the participants can be found in Table 6.

### Results

The mean insulin level at the end of the first phase insulin response to glucose was 99.84 mU/L instead of the normal limits of 3-25 mU/L. The maximum level of insulin was 940.9 mU/L, which was observed in newly diagnosed patients, and a minimum of 6.8 mU/L, seen in patients with β-cell exhaustion. The insulin standard deviation was 118.87 m U/L.

The mean level of proinsulin was 71.75 pmol/L at the end of the first phase insulin response to glucose. The maximum was 840 pmol/L, seen in newly diagnosed patients and those with early β-cell exhaustion, and a minimum of 1 pmol/L was seen in those with severe β-cell exhaustion/burn out and early diabetes. The proinsulin standard deviation was found to be pmol/L.

TABLE 3
Dependent Variables: Descriptions and Measurements

| | Description of | |
|---|---|---|
| Dependent variable | variable | Measurement |
| First phase serum insulin | Serum insulin obtain | Insulin measured in |
| | at 15 min after oral | mU/L |
| | challenge during | |
| | OGIST | |
| First phase proinsulin | Serum proinsulin | Proinsulin measured |
| | obtain at 15 min after | in pmol/L |
| | oral challenge during | |
| | OGIST | |
| First phase C-peptide | Serum C-peptide | C-peptide measured |
| | obtain at 15 min after | in ng/mL |
| | oral challenge during | |
| | OGIST | |
| Hemoglobin A1c | Glycated Hemoglobin | Measured as % of |
| | (Hb A1c), provides | glycated hemoglobin |
| | an index of the | |
| | average blood sugar | |
| | for a 2-4 month | |
| Serum triglycerides | period | Triglycerides |
| | Complex lipids of | measured in mg/dl |
| | esterified glycerol | |
| | influenced by insulin | |

The mean C-peptide levels at the end of the first phase insulin response to glucose was 5.26 ng/ml with the maximum levels at 26 and minimum C-peptide level 0 pmol/L. The C-peptide standard deviation was 3.24 pmol/L. C-peptide levels at the end of the first phase insulin response to glucose should always be slightly higher than normal due to its secretion through the kidneys 30-40 minutes after release.

Normal glucose levels at the end of the first phase insulin response to glucose are 80-120 mg/dl. The mean glucose levels in this study were 147.46 mg/dl with the maximum 452 mg/dl and minimum 71 mg/dl with SD of 53.13 mg/dl. The higher levels of glucose were seen in those patients with higher HbA1c levels and lower C-peptide levels suggesting early onset diabetes and severe β-cell exhaustion.

TABLE 4
Independent Variables: Descriptions and Measurements

| Independent | Description of | Level of |
|---|---|---|
| variables | variable | measurement |
| Glucose (Oral) | Oral glucose is | Measured in |
| | given during | grams of actual |
| | OGIST | glucose |
| Age | Age of patients | Continuous |
| Gender | Gender of patients | Categorical; Male |
| | | or Female |
| Ethnicity | Ethnic group | Categorical; |
| | stated by patient | Caucasian, Black, |
| | | Hispanic, Asian, |
| | | American Indian, |
| | | Other |
| BMI | Body Mass Index. | Continuous |
| | Body mass divided | |
| | by the square of | |
| | the body height | |
| | expressed as units | |
| | of kg/m^{2.} | |

Normal HbA1c levels are below 5.6%, with 5.7-5.9% being considered prediabetes with early β-cell exhaustion and 6.0% and above are classified as diabetic. In this study the mean HbA1c was 5.9% (prediabetes) with the maximum 13.8% (uncontrolled diabetes), and minimum of 3.5% with SD 1.53%. The higher levels of HbA1c were seen with β-cell exhaustion.

TABLE 5
Summary of Data Analyses with Comparative Surrogate Tests

| Variable | Analysis | Surrogate test |
|---|---|---|
| Insulin | Binomial logistic regression | OGIST/HOMA/McA/QUICKI |
| Proinsulin | Binomial logistic regression | OGIST |
| Glucose | Binomial logistic regression | OGIST/HOMA/QUICKI |
| HbA1c | Binomial logistic regression | OGIST |
| C-peptide | Binomial logistic regression | OGIST |
| Triglycerides | Binomial logistic regression | Comparative study with |
| | | surrogate test McAuley's |
| | | index (McA) |

Triglycerides cholesterol is produced by the liver and used for cell wall production and energy for certain cells of the body. Insulin is the feedback hormone for triglyceride production. In this study the mean triglyceride level was 186.9 mg/dl with the maximum of 696 mg/dl and minimum level at 32 mg/dl with SD 1.86 mg/dl. Newly diagnosed patients with IR demonstrated higher levels of triglycerides with the lower levels found in those patients that were well treated.

One sample t-test (Table 7) was conducted to evaluate the dependent variables of the OGIST results mean against the null hypothesis. The mean results were then compared to the normal values for each dependent variable (e.g.,) glucose, insulin, proinsulin, C-peptide, and HbA1c. The null hypothesis H₀1 was rejected because p<0.000 was observer for all the dependent variables.

TABLE 6
Descriptive Statistics of Patients Tested for Insulin Resistance Via OGIST

| | N | Minimum | Maximum | M | SD | Kurtosis | SE |
|---|---|---|---|---|---|---|---|
| Age | 250 | 12.0 | 79.0 | 50.13 | 14.55 | −.301 | .307 |
| BMI | 251 | 3.37 | 468.29 | 240.47 | 162.69 | −1.69 | .306 |
| Insulin (1 U/L) | 251 | 6.8 | 940.9 | 95.28 | 116.51 | 24.40 | .306 |
| Glucose (1 g/dL) | 251 | 71.0 | 452.0 | 147.85 | 52.08 | 6.15 | .306 |
| HgA1c (%) | 251 | 3.5 | 138.0 | 6.42 | 8.46 | 236.29 | .306 |
| Triglycerides | 251 | 4.8 | 961.0 | 163.58 | 128.68 | 11.22 | .306 |
| (1 g/dL) | | | | | | | |
| HOMA | 251 | 3.56 | 1520.24 | 330.0 | 261.49 | .842 | .306 |
| QUICKI | 251 | .159 | .274 | .188 | .022 | 2.425 | .300 |
| McA | 251 | .587 | 9.05 | 1.35 | 1.38 | 15.66 | .300 |
| Valid N | 250 | | | | | | |
| (listwise) | | | | | | | |

TABLE 7
One Sample t Test

| | t | df | p (2-tailed) | Mean Difference | Lower | Upper |
|---|---|---|---|---|---|---|
| Insulin (mU/L) | 12.57 | 223 | .000 | 99.84 | 84.18 | 115.49 |
| Proinsulin (pmol/L) | 12.34 | 222 | .000 | 71.75 | 60.30 | 83.21 |
| C-Peptide (ng/mL) | 24.08 | 222 | .000 | 5.23 | 4.81 | 5.66 |
| Glucose (mg/dL) | 41.54 | 223 | .000 | 147.46 | 140.46 | 154.46 |
| HbA1c (%) | 57.78 | 223 | .000 | 5.91 | 5.71 | 6.11 |
| Triglycerides (mg/dL) | 25.66 | 223 | .000 | 186.92 | 172.57 | 201.27 |
| HOMA | 9.82 | 223 | .000 | 11.30 | 9.037 | 13.57 |
| QUICKI | 162.26 | 223 | .000 | .25 | .25 | .25 |
| McA | 46.30 | 223 | .000 | 4.07 | 3.90 | 4.24 |

### Analysis

Binomial logistic regression analysis was conducted to evaluate the relationship of insulin, proinsulin, and C-peptide with the independent variables. Because the dependent variables demonstrated a dichotomous tendency and did not meet the criteria assumptions for linear regression, binomial logistic regression was used in this study. The first step was to create a binary categorical dependent variable for each continuous variable. Each dependent variable was changed from continuous to categorical binary variables by using the median value of each group as a cutoff point and separating into two groups above and below the median value. This allowed the dependent variables to meet assumption testing for binomial logistic regression. This information was then used to determine if the OGIST is capable of predicting insulin resistance in the patient meeting the criteria for metabolic syndrome. Tables 3-15 demonstrate the results of this analysis. Bonferroni multiple-comparison correction was used since multiple tests were performed simultaneously resulting in statistical significance being accepted with p<0.05.

Binomial logistic regression was used to evaluate the relationship between the predictors and the dependent variable insulin Table 8.

TABLE 8
Binomial Regression Analysis Summary: Dependent Variable-Insulin

| | | | | | | | | 95% C.I. for OR | |
|---|---|---|---|---|---|---|---|---|---|
| | | B | S.E. | Wald | df | p | OR | Lower | Upper |
| Step | In_age | −2.60 | 1.14 | 5.15 | 1 | .023 | .074 | .008 | .700 |
| 1^{a} | In_BMI | 1.07 | 1.58 | .45 | 1 | .500 | 2.915 | .130 | 65.18 |
| | Ln_Ethnicity | −1.97 | 1.16 | 2.85 | 1 | .091 | .139 | .014 | 1.37 |
| | Ln_HOMA | −47.23 | 14.98 | 9.93 | 1 | .002 | .000 | .000 | .000 |
| | Ln_McA | −5.00 | 1.84 | 7.36 | 1 | .007 | .007 | .000 | .249 |
| | Ln_QUICKI | −513.38 | 152.44 | 11.34 | 1 | .001 | .000 | .000 | .000 |
| | Constant | −600.89 | 179.50 | 11.20 | 1 | .001 | .000 | | |

^{a}Variable(s) entered on Step 1: In_age, In_BMI, Ln_Ethnicity, Ln_HOMA, Ln_McA, Ln_QUICKI.

The results in Table 8 demonstrate the model is statistically significant, x²(6)=231.548, p<0.005. Nagelkerke R² was 87.1% and the model correctly identified 95.2% of the cases. Sensitivity of the test was 91.4% and specificity was 96.6%. Positive predictive value was 91.4% with a negative predictive value of 96.6%. The predictor variables used in this model included age, BMI, ethnicity, HOMA, McA and QUICKI. Out of these variables age, HOMA and QUICKI, and McA were found to be significant. This analysis demonstrates that the odds or reduced insulin sensitivity in older adults is 7.4% higher than younger adults. The significance of the surrogate tests for insulin resistance compared to the OGIST were HOMA: 0.002, McA: 0.007, and QUICKI: 0.001

Binomial logistic regression was used to evaluate the relationship between the predictors and the dependent variable proinsulin in table 9.

TABLE 9
Binomial Regression Analysis Model Summary: Dependent Variable-Proinsulin

| | | | | | | | | 95% C.I. for OR | |
|---|---|---|---|---|---|---|---|---|---|
| | | B | S.E. | Wald | df | p | OR | Lower | Upper |
| Step | In_age | .247 | .43 | .32 | 1 | .568 | 1.280 | .548 | 2.991 |
| 1^{a} | In_BMI | 1.089 | .75 | 2.06 | 1 | .151 | 2.971 | .672 | 13.140 |
| | Ln_Ethnicity | −.109 | .52 | .04 | 1 | .834 | .897 | .323 | 2.491 |
| | Ln_HOMA | −7.961 | 3.13 | 6.44 | 1 | .011 | .000 | .000 | .163 |
| | Ln_McA | −.650 | .76 | .71 | 1 | .397 | .522 | .116 | 2.347 |
| | Ln_QUICKI | −84.349 | 29.67 | 8.07 | 1 | .004 | .000 | .000 | .000 |
| | Constant | −103.842 | 34.87 | 8.86 | 1 | .003 | .000 | | |

Variable(s) entered on step 1: In_age, In_BMI, Ln_Ethnicity, Ln_HOMA, Ln_McA, Ln_QUICKI.

The results in Table 9 demonstrates the model is statistically significant, x²(6)=67.665, p<0.005. Nagelkerke R² was 31.8% and the model correctly identified 70.7% of the cases. Sensitivity of the test was 71.8% and specificity was 69.7%. Positive predictive value was 67.7% with a negative predictive value of 73.6%. The predictor variables used in this model included age, BMI, Ethnicity, HOMA, McA and QUICKI. Age, BMI, and ethnicity did not demonstrate any significance in proinsulin production. The significance of the surrogate tests for proinsulin compared to the OGIST were HOMA: 0.011 and QUICKI: 0.004. Binomial logistic regression was used to evaluate the relationship between the predictors and the dependent variable C-peptide Table 10.

The results in Table 10 demonstrate the model is statistically significant, x²(6)=116.545, p<0.005. Nagelkerke R² was 49.8% and the model correctly identified 81.1% of the cases. Sensitivity of the test was 83.2% and specificity was 79.0%. Positive predictive value was 80% with a negative predictive value of 82.3%. Age, BMI, and ethnicity did not demonstrate any significance in C-peptide. The significance of the surrogate tests for C-peptide compared to the OGIST were HOMA: 0.001, McA: 0.006, and QUICKI: 0.001. Binomial logistic regression was used to evaluate the relationship between the predictors and the dependent variable HbA1c Table 11.

TABLE 10
Binomial Regression Analysis Model Summary: Dependent Variable-C-peptide

| | | | | | | | | 95% C.I. for OR | |
|---|---|---|---|---|---|---|---|---|---|
| | | B | S.E. | Wald | df | p | OR | Lower | Upper |
| Step | In_age | −.62 | .51 | 1.47 | 1 | .225 | .536 | .196 | 1.467 |
| 1^{a} | In_BMI | −.39 | .87 | .197 | 1 | .657 | .677 | .121 | 3.792 |
| | Ln_Ethnicity | −.58 | .62 | .851 | 1 | .356 | .560 | .163 | 1.921 |
| | Ln_HOMA | −12.63 | 3.93 | 10.309 | 1 | .001 | .000 | .000 | .007 |
| | Ln_McA | −2.45 | .89 | 7.585 | 1 | .006 | .086 | .015 | .493 |
| | Ln_QUICKI | −129.71 | 37.50 | 11.96 | 1 | .001 | .000 | .000 | .000 |
| | Constant | −145.12 | 43.91 | 10.92 | 1 | .001 | .000 | | |

^{a}Variable(s) entered on step 1: In_age, In_BMI, Ln_Ethnicity, Ln_HOMA, Ln_McA, Ln_QUICKI.

The results in Table 11 demonstrate the model is statistically significant, x²(6)=36.34, p<0.0005. Nagelkerke R² was 18.2% and the model correctly identified 66.7% of the cases. Sensitivity of the test was 77.5% and specificity was 53.2%. Positive predictive value was 67.29% with a negative predictive value of 65.5%. The predictor variables used in this model included age, BMI, and ethnicity. Both age and ethnicity demonstrated significance in HbA1c. The significance of the surrogate tests for HbA1c compared to the OGIST was HOMA: 0.032.

TABLE 11
Binomial Regression Analysis Model Summary: Dependent Variable-HbA1c

| | | | | | | | | 95% C.I. for OR | |
|---|---|---|---|---|---|---|---|---|---|
| | | B | S.E. | Wald | df | p | OR | Lower | Upper |
| Step | In_age | 1.86 | .44 | 17.44 | 1 | .000 | 6.46 | 2.692 | 15.509 |
| 1^{a} | In_BMI | 1.18 | .73 | 2.57 | 1 | .109 | 3.25 | .770 | 13.797 |
| | Ln_Ethnicity | 1.83 | .60 | 9.25 | 1 | .002 | 6.27 | 1.922 | 20.500 |
| | Ln_HOMA | 5.43 | 2.53 | 4.59 | 1 | .032 | 229.71 | 1.594 | 33105.192 |
| | Ln_McA | .71 | .734 | .950 | 1 | .330 | 2.04 | .485 | 8.609 |
| | Ln_QUICKI | 43.00 | 22.62 | 3.61 | 1 | .057 | 47.5 | .263 | 8.59 |
| | Constant | 34.004 | 25.86 | 1.72 | 1 | .189 | 58.60 | | |

^{a}Variable(s) entered on step 1: In_age, In_BMI, Ln_Ethnicity, Ln_HOMA, Ln_McA, Ln_QUICKI.

TABLE 12
Binomial Regression Analysis Validation Model Summary: Dependent Variable Triglycerides

| | | | | | | | | 95% C.I. for OR | |
|---|---|---|---|---|---|---|---|---|---|
| | | B | S.E. | Wald | df | p | OR | Lower | Upper |
| Step | In_age | −2.704 | .756 | 12.804 | 1 | .000 | .067 | .015 | .294 |
| 1^{a} | In_BMI | 1.119 | 1.357 | .680 | 1 | .409 | 3.062 | .214 | 43.720 |
| | Ln_Ethnicity | −2.627 | 1.182 | 4.938 | 1 | .026 | .072 | .007 | .734 |
| | Ln_HOMA | −7.514 | 5.176 | 2.107 | 1 | .147 | .001 | .000 | 13.902 |
| | Ln_McA | 22.945 | 3.380 | 46.082 | 1 | .000 | 92271826 | 12243677.52 | 6953866483000.00 |
| | Ln_QUICKI | −111.22 | 46.514 | 5.718 | 1 | .017 | .000 | .000 | .000 |
| | Constant | −158.93 | 54.879 | 8.387 | 1 | .004 | .000 | | |

Variable(s) entered on step 1: In_age, In_BMI, Ln_Ethnicity, Ln_HOMA, Ln_McA, Ln_QUICKI.

Binomial logistic regression was used to evaluate the relationship between the predictors and the dependent variable triglycerides. Table 12 shows the comparison of the surrogate test McA with OGIST.

The results in Table 12 demonstrate the model is statistically significant, x²(6)=36.34, p<0.0005. Nagelkerke R² was 78.4% and the model correctly identified 92.4% of the cases. Sensitivity of the test was 94.6% and specificity was 89.9%. Positive predictive value was 91.1% with a negative predictive value of 93.8%. The predictor variables used in this model included age, BMI, and ethnicity. HOMA, McA and QUICKI were surrogate predictors. Out of these variables only age and ethnicity demonstrated a correlation with elevated triglycerides. The significance of the surrogate tests for triglycerides compared to the OGIST were McA: 0.001 and QUICKI: 0.017.

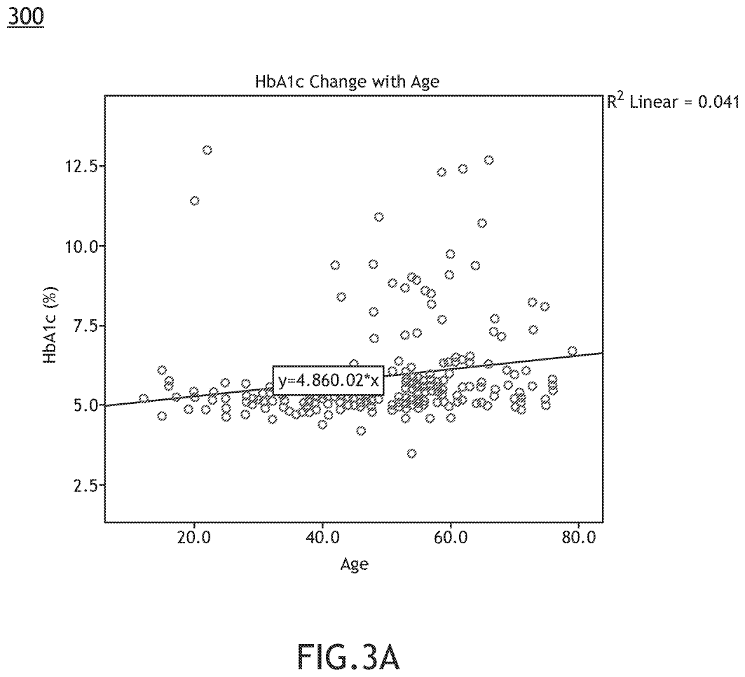

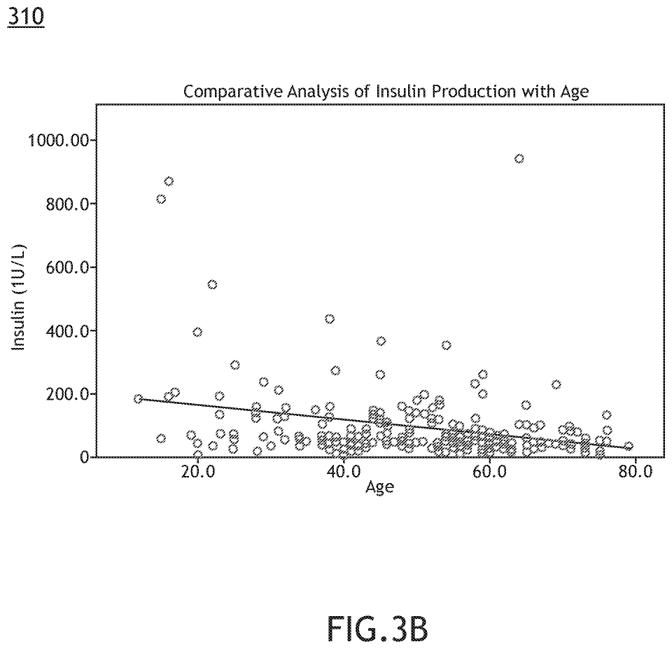

Scatter plot graph analysis was completed on insulin, proinsulin, C-peptide, and HbA1c. The dependent variables insulin, proinsulin, C-peptide, and HgA1c were placed on the X-axis with age placed on the Y-axis for possible differences seen during advanced age. FIG. 3A is a scatterplot analysis 300 illustrating the relationship between HbA1c and age. HbA1c levels increased with increased age in those patients diagnosed with IR. The higher HbA1c levels found in patients over 40 years of age is due to advanced β-cell exhaustion due to IR. The higher HbA1c levels observed in younger patients was observed with severe IR. Those patients having elevated insulin levels above 500 mU/L at the end of the first phase insulin response to glucose.

FIG. 3B is a scatterplot analysis 310 illustrating the relationship between insulin production and age. Binomial logistic regression analysis was completed to confirm the changes seen in insulin production with age. To accommodate the requirements for binomial regression patient's ages were divided into those under 50 years of age and those 50 years of age and older.

The results in Table 13 demonstrate the model is statistically significant, x2(4)=23.14, p<0.0005. Nagelkerke R2 was 11.9% and the model correctly identified 64.3% of the cases. Sensitivity of the test was 71.2% and specificity was 58.0%. Positive predictive value was 60.4% with a negative predictive value of 69%. The predictor variables used in this model were InsulinCat, ProinsulinCat, CPeptideCat, and HbA1cCat. Only InsulinCat and HgA1cCat were found to be significant with InsulinCat: 0.002 and HbA1cCat: 0.001.

TABLE 13
Binomial Regression Analysis Validation
Model Summary: Dependent Variable Age

| | | | | | | | | 95% C.I. for OR | |
|---|---|---|---|---|---|---|---|---|---|
| | | B | S.E. | Wald | df | p | OR | Lower | Upper |
| Step | InsulinCat | 1.130 | .364 | 9.625 | 1 | .002 | 3.095 | 1.516 | 6.319 |
| 1^{a} | ProinsulinCat | −.220 | .309 | .510 | 1 | .475 | .802 | .438 | 1.469 |
| | CPeptideCat | −.154 | .329 | .220 | 1 | .639 | .857 | .450 | 1.633 |
| | HbA1cCat | −.898 | .274 | 10.721 | 1 | .001 | .407 | .238 | .697 |

^{a}Variable(s) entered on step 1: InsulinCat, ProinsulinCat, CPeptideCat, HbA1cCat.

The OGIST is designed to evaluate insulin sensitivity by directly measuring the patients' insulin, proinsulin, C-peptide, and glucose levels at the end of the first phase insulin response to glucose. An oral glucose challenge of 36 grams is given is initially given to the patient with labs being obtained 15 minutes later. HbA1c is also obtained to evaluate the patient's glucose control. This allows evaluation of insulin production by direct measure of C-peptide at the end of the first phase insulin response to glucose and glucose homeostasis by HbA1c. Insulin sensitivity is then evaluated by direct measure of insulin and proinsulin at the end of the first phase insulin response to glucose. This test also allows the evaluation of the pancreatic β-cells function/exhaustion by the measurement of insulin, proinsulin, C-peptide, and comparing with HgA1c levels.

A total 251 patients were included into the study. A total of 26 patient's data were not considered for the study due to the lack of critical data. The mean study patient BMI was found to be higher than that of the normal population (34 vs 28) which would be expected with the increased insulin production. Mean insulin levels in this study at the end of the first phase insulin response was 99.84 mU/L compared to normal levels of 3-25 mU/L. The higher level of insulin was found to be in a newly diagnosed patient with 940.9 mU/L and a low of 6.8 mU/L which was observed in a patient with prediabetes and β-cell exhaustion. C-peptide levels should normally be elevated at the end of the first phase insulin response due to the clearance of this peptide by the kidneys between 30 and 40 minutes. The C-peptide levels in this study ranged between 0 pmol/L and 26 pmol/L with mean levels at 5.26 pmol/L. Lower than normal levels were seen in those patients with β-cell exhaustion and diabetes with no or very poor insulin production. The higher C-peptide levels were found in patients with uncontrolled IR producing very high levels of insulin. Glucose levels were found to parallel HgA1c levels and based primarily on the patients' glucose and HgA1c homeostasis. Triglycerides were found to be higher in patients diagnosed with IR and higher insulin levels. The mean triglyceride level in the study was 189.6 mg/dl with the minimum of 32 mg/dl in patients being treated for IR and a high of 696 mg/dl found in patients newly diagnosed with higher insulin levels.

When comparing the OGIST to HOMA, QUICKI, and McA, HOMA had similar predictive quality to OGIST for insulin and C-peptide alone. The QUICKI test demonstrated similar predictive qualities to OGIST for insulin, proinsulin, and C-peptide. McA demonstrated similar predictive qualities to OGIST for triglycerides only. OGIST demonstrated significant predictive qualities for insulin, proinsulin, C-peptide, HbA1c, and triglycerides. When evaluating the ability of the tests to evaluate the individuals' insulin sensitivity, production and β-cell function OGIST was superior to the other tests. The OGIST superiority could be due to the ability of the test to evaluate the combination of insulin, proinsulin, glucose, C-peptide, and HgA1c together at the end of the first phase insulin response to glucose. These results demonstrate that the OGIST could predict IR in the individual at the end of the first phase insulin response to glucose by measuring serum insulin, proinsulin, C-peptide, glucose, and HbA1c levels. The OGIST was effective in determining insulin sensitivity within the individual and β-cell function.

The OGIST can predict insulin resistance in a clinical setting without invasive hospital testing as seen in the glucose clamp technique. The OGIST allows the direct measurement of many insulin indices, including insulin, proinsulin, C-peptide, and HbA1c, at the end of the first phase insulin response to glucose. The OGIST allows evaluation of both the binding of insulin to target tissues and insulin production of the pancreatic β-cells. The ability of the OGIST to identify insulin sensitivity, beta cell function, and the progression of the individual to diabetes makes OGIST making this a valuable test in identifying and treating insulin resistance and reducing Type 2 diabetes.

In some cases, the methods disclosed herein involve comparing levels or occurrences to a reference. The reference can take on a variety of forms. In some cases, the reference comprises predetermined values for the plurality of analytes (e.g., each of the plurality of analytes). The predetermined value can take a variety of forms. It can be a level or occurrence of an analyte obtained from a subject known to have insulin resistance or diabetes (e.g., a symptomatic subject), or obtained from a subject known not to suffer from insulin resistance or known to not have a diabetes (e.g., an asymptomatic subject). It can be a level or occurrence in the same subject, e.g., at a different time point. A predetermined level can be single cut-off value, such as a median or mean. It can be a range of cut-off (or threshold) values, such as a confidence interval. It can be established based upon comparative groups, such as where the risk in one defined group is a fold higher, or lower, (e.g., approximately 2-fold, 4-fold, 8-fold, or more) than the risk in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk. Moreover, the reference could be a calculated reference, most preferably the average or median, for the relative or absolute amount of an analyte of a population of individuals comprising the subject to be investigated.

It should be understood that this disclosure is not limited to the particular methodology, protocols, and reagents, etc., described herein and as such may vary. The terminology used herein is for the purpose of describing particular embodiments only, and is not intended to limit the scope of the present disclosure. It is also to be understood that embodiments of the methods disclosed herein may include one or more of the steps described herein. Further, such steps may be carried out in any desired order and two or more of the steps may be carried out simultaneously with one another. Two or more of the steps disclosed herein may be combined in a single step, and in some embodiments, one or more of the steps may be carried out as two or more sub-steps. Further, other steps or sub-steps may be carried in addition to, or as substitutes to one or more of the steps disclosed herein.

A number of embodiments of the invention have been described. Nevertheless, it will be understood that various modifications may be made without departing from the spirit and scope of the disclosure. Accordingly, other embodiments are within the scope of the following claims.

What is claimed is:

1. A method of diagnosing and treating insulin resistance at an end of a first phase insulin response to glucose in a mammalian subject comprising:
administering an amount of glucose to the mammalian subject capable of producing a first phase insulin response to glucose;
obtaining a blood sample from the mammalian subject at the end of the first phase insulin response to glucose;
isolating a serum sample from the blood sample;
determining a serum concentration of at least one biomarker for insulin resistance from a group of biomarkers comprising insulin, proinsulin, C-peptide, glucose, and hemoglobin A1c in the serum sample;
performing an analysis of the serum concentration of the at least one biomarker for insulin resistance in the serum sample to determine if the mammalian subject is insulin resistant, wherein the analysis includes comparing the determined concentration of the at least one biomarker in the serum sample to insulin resistance-positive and insulin resistance-negative reference concentrations; and
administering a treatment to the mammalian subject if the mammalian subject is determined to be insulin resistant, wherein the treatment comprises at least one of weight loss, dietary modification, exercise, or drug therapy, wherein the drug therapy is an effective amount of at least one of an insulin, a metformin, a thiazolidinedione, or a sulfonylurea.

2. The method of claim 1, wherein the concentrations of at least one of insulin or proinsulin in the serum sample are analyzed to determine binding of insulin to target tissues in the mammalian subject, wherein an effective treatment is administered to the mammalian subject based upon the analysis, wherein the effective treatment comprises at least one of weight loss, dietary modification, exercise, or drug therapy, wherein the drug therapy is an effective amount of at least one of an insulin, metformin, thiazolidinedione, or sulfonylurea.

3. The method of claim 1, wherein the concentrations of at least one of insulin, C-peptide and proinsulin in the serum sample are analyzed to determine pancreatic β-cell production of insulin in the mammalian subject, wherein an effective treatment is administered to the mammalian subject based upon the analysis, wherein an effective treatment comprises at least one of weight loss, dietary modification, exercise, or drug therapy, wherein the drug therapy is an effective amount of at least one of insulin, metformin, thiazolidinedione, or sulfonylurea.

4. The method of claim 3, wherein the analysis of at least one of insulin, C-peptide, and proinsulin further comprises a comparison to hemoglobin A1c levels.

5. The method of claim 1, wherein the serum sample is collected at least 15 minutes after the administration of glucose.

6. The method of claim 1, wherein the mammalian subject fasts at least 8 hours before the glucose is administered.

7. The method of claim 1, wherein the biomarkers further comprise indicators of diseases associated with insulin resistance.

8. The method of claim 7, wherein the disease associated with insulin resistance is liver damage, wherein the biomarkers for liver damage comprise at least one of an alanine transaminase, an aspartate aminotransferase, an alkaline phosphatase or free fatty acids.

9. The method of claim 7, wherein the disease associated with insulin resistance is cardiovascular disease, wherein the biomarkers for cardiovascular disease comprises at least one of C-reactive protein, BNP, Lipoprotein(a), total cholesterol, LDL, HDL, Triglycerides or non-HDL cholesterol.

10. The method of claim 7, wherein the disease associated with insulin resistance is inflammation, wherein the biomarkers for inflammation comprise at least one of PAI-1, C-reactive protein, ferritin, fibrinogen, homocysteine, interleukin-6, or Lipoprotein(a).

11. A method of diagnosing insulin resistance at an end of a first phase insulin response to glucose in a mammalian subject capable of producing a first phase insulin response to glucose comprising:
isolating a serum sample from a blood sample obtained at the end of the first phase insulin response to glucose from the mammalian subject administered with an amount of glucose;
determining a serum concentration of at least one biomarker for insulin resistance from a group of biomarkers consisting of insulin, proinsulin, C-peptide, glucose, and hemoglobin A1c in the serum sample; and
performing an analysis of the serum concentration of the at least one biomarker for insulin resistance in the serum sample to determine if the mammalian subject is insulin resistant, wherein the analysis includes comparing the determined concentration of the at least one biomarker in the serum sample to insulin resistance-positive and insulin resistance-negative reference concentrations.

12. The method of claim 11, wherein the concentrations of at least one of insulin or proinsulin in the serum sample are analyzed to determine binding of insulin to target tissues in the mammalian subject.

13. The method of claim 11, wherein the concentrations of at least one of insulin, C-peptide and proinsulin in the serum sample are analyzed to determine pancreatic β-cell production of insulin in the mammalian subject.

14. The method of claim 13, wherein the analysis of at least one of insulin, C-peptide, and proinsulin further comprises a comparison to hemoglobin A1c levels.

15. The method of claim 11, wherein the serum sample is collected at least 15 minutes after the administration of glucose.

16. The method of claim 11, wherein the mammalian subject fasts at least 8 hours before the glucose is administered.

17. The method of claim 11, wherein the biomarkers further comprise indicators of diseases associated with insulin resistance.

18. The method of claim 17, wherein the disease associated with insulin resistance is liver damage, wherein the biomarkers for liver damage comprise at least one of an alanine transaminase, an aspartate aminotransferase, an alkaline phosphatase or free fatty acids.

19. The method of claim 17, wherein the disease associated with insulin resistance is cardiovascular disease, wherein the biomarkers for cardiovascular disease comprises at least one of C-reactive protein, BNP, Lipoprotein (a), total cholesterol, LDL, HDL, Triglycerides or non-HDL cholesterol.

20. The method of claim 17, wherein the disease associated with insulin resistance is inflammation, wherein the biomarkers for inflammation comprise at least one of PAI-1, C-reactive protein, ferritin, fibrinogen, homocysteine, interleukin-6, or Lipoprotein(a).
